(19) United States
(12) Patent Application Publication
(10) Pub. No.: US 2026/0219346 A1
(43) Pub. Date: Jul. 30, 2026

(54) MAGNETIC RESONANCE SEQUENCE WITH FREE EXCITATION PULSE AND FORM-BOUND REFOCUSING PULSES

(71) Applicant: Siemens Healthineers AG, Forchheim (DE)

(72) Inventor: Jürgen Herrler, Erlangen (DE)

(21) Appl. No.: 19/449,977

(22) Filed: Jan. 15, 2026

(30) Foreign Application Priority Data
Jan. 28, 2025 (EP) 25154397

(51) Int. Cl. G01R 33/561; A61B 5/055; G01R 33/24; G01R 33/44; G01R 33/54

(52) U.S. Cl. CPC G01R 33/5617; A61B 5/055; G01R 33/246; G01R 33/443; G01R 33/543; G01R 33/5612

(57) ABSTRACT

Systems and methods for provision of a magnetic resonance sequence. In a method, a magnetic field distribution dataset is provided. An excitation pulse is established that includes an excitation pulse shape. The excitation pulse shape is established as a function of the magnetic field distribution dataset. A refocusing pulse shape is further predetermined. At least one refocusing pulse is established, which has the predetermined refocusing pulse shape. The magnetic resonance sequence, which includes the excitation pulse and the at least one refocusing pulse may be provided in the form of a sequence dataset.

### CROSS REFERENCE TO RELATED APPLICATIONS

This application claims the benefit of EP 25154397.1 filed on Jan. 28, 2025, which is hereby incorporated by reference in its entirety.

### FIELD

Embodiments relate to a method for provision of a magnetic resonance sequence, to a magnetic resonance apparatus and to a computer program product.

### BACKGROUND

In medical engineering, imaging by magnetic resonance (MR), also called magnetic resonance imaging (MRI), is characterized by high soft tissue contrast. In such cases an examination object, for example a patient, is positioned in an examination area of a magnetic resonance apparatus, in which a main magnet of the magnetic resonance apparatus generates a main magnetic field. During a magnetic resonance measurement in accordance with a magnetic resonance sequence, radio-frequency (RF) transmit pulses for generating a magnetic alternating field (also called a B1 field) and gradient pulses for generating a magnetic field gradient, which are superimposed on the main magnetic field, are irradiated into the examination area and thus nuclear spins are excited in the examination object. Spatially encoded echo signals are triggered by this in the examination object, that are often also called magnetic resonance signals. The magnetic resonance signals may be used for reconstruction of magnetic resonance images.

It is precisely with higher strengths of the main magnetic field, for example with 7 tesla, that the resulting magnetic fields may exhibit a comparatively high inhomogeneity, that may manifest itself as artifacts in the magnetic resonance images. In order to reduce this problem, the RF transmit pulses for example may be adapted in order to generate a B1 field of high homogeneity. However, the calculation of such adapted RF transmit pulses may be very time-consuming. Further, a part of the RF power irradiated in is absorbed by the examination object; this is described by the specific absorption rate (SAR). In order not to endanger the patient, the SAR may not exceed specific limit values.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide a method for provision of a magnetic resonance sequence with which magnetic resonance signals for generation of high-quality magnetic resonance images may be acquired efficiently.

Accordingly a computer-implemented method for provision of a magnetic resonance sequence, for example of a spin echo sequence, is. In this method a magnetic field distribution dataset is provided. Moreover, an excitation pulse is established, that has an excitation pulse shape, wherein the excitation pulse shape is established as a function of the magnetic field distribution dataset. A refocusing pulse shape is further predetermined. At least one refocusing pulse is established, that has the predetermined refocusing pulse shape. The magnetic resonance sequence, that includes the excitation pulse and the, for example chronologically consecutive, at least one refocusing pulse, is provided, for example in the form of a sequence dataset.

Excitation pulses and refocusing pulses are usually RF transmit pulses, that may be sent out by a radio-frequency antenna unit of a magnetic resonance apparatus. The radio-frequency antenna unit may include one or more transmit coils, for example transmit coil elements, to each of which a transmit channel may be assigned.

The magnetic resonance sequence may be an activation sequence, that for example is selective for a destination region, for example a slice, for producing a destination excitation state, for example a specific, homogeneous flip angle of the spins of the examination object, for a detection process of magnetic resonance signals of an examination object with a magnetic resonance apparatus. In this case the magnetic resonance sequence includes radio-frequency pulses, for example excitation pulses and refocusing pulses, to be output over at least one transmit channel, for example over a number of transmit channels, of a radio frequency antenna arrangement.

Spin echo sequences are widely used sequence types in the field of MRI. A spin echo sequence usually includes a series consisting of an excitation pulse, for example a 90° pulse, and at least one refocusing pulse, for example a 180° pulse. A spin echo sequence may for example be used for generating T1-weighted, proton-dense-weighted or strongly T2-weighted magnetic resonance images.

What is typically utilized in a spin echo sequence is that a magnetic resonance signal reappears after a decay of an FID signal. To do this, a dephasing of a nuclear spin (transverse magnetization decay) is canceled by radiating in a refocusing pulse. The spins go back into phase, and the spin echo arises at the point in time TE (echo time).

The magnetic field distribution dataset may for example include a B1 field map, or B1 map for short. The magnetic field distribution dataset may also include a B0 field map, or B0 map for short.

The B1 map usually describes a spatial distribution of a B1 field. A B1 map is preferably an, for example spatial, representation of a B1 field. The B1 field is usually the magnetic alternating field of the RF irradiation generated by a transmit coil of a magnetic resonance apparatus. The B0 map usually describes a spatial distribution of a B0 field. A B1 map is preferably an, for example spatial, representation of a B0 field. The B0 field is usually the static main magnetic field of the magnetic resonance apparatus. The B0 field is usually generated by an, often superconducting, main magnet of the magnetic resonance apparatus. A B1 field or a B0 field may for example be represented by an image in which the intensity of an image pixel or image voxel represents the strength of the B1 field or of the B0 field at the location of the image pixel or image voxel.

Advantageously the B1 and/or the B0 field describe the respective field distribution when the examination object is located in an examination area of the magnetic resonance apparatus, for example in a measurement position. With higher magnetic field strengths for example, greater inhomogeneities of the fields may arise through the presence of the examination object. Advantageously such inhomogeneities may be reduced by specially adapted RF pulses, for example excitation pulses and/or refocusing pulses.

For generating the magnetic field distribution dataset a magnetic resonance measurement may be carried out, for example applied, with which magnetic resonance signals are recorded, with the aid of which the magnetic field distribution dataset, for example a B1 and/or B0 map, may be generated. A series of methods for generation of B1 and/or B0 maps are known.

The excitation pulse shape is the pulse shape of the excitation pulse. The refocusing pulse shape is the pulse shape of the at least one refocusing pulse. The remarks below about the term “pulse shape” relate for example to the excitation pulse shape and/or refocusing pulse shape. The remarks below about the term “pulse” relate for example to the excitation pulse and/or refocusing pulse.

A pulse shape (i.e. for example excitation pulse shape or refocusing pulse shape) is for example the profile and/or the shape of a pulse (i.e. for example excitation pulse or refocusing pulse). A pulse shape may for example be described by an envelope of the pulse, for example in the base band. The pulse shape is for example independent of an absolute height, for example amplitude, and/or an absolute width, for example length, of the pulse.

A pulse shape of a pulse may for example be described by a temporal course of the pulse, for example expressed as a pulse profile S(t), wherein the pulse profile S(t) may be described for example by a complex-value function and t represents the time.

For example, it is possible for a pulse shape of a pulse to be able to be described by a temporal course of an, for example real-value, momentary value and/or a deflection of the pulse and/or a phase of the pulse. For example, functions of pulses, for example sinc pulses, may have negative values, that may then be represented however as positive amplitude values with 180° phase offset in the course of the phase.

The pulse shape, for example its amplitude and/or width, is standardized. The pulse shape may be a standardized RF pulse shape. This may be expressed mathematically for example with S(t)=nS(t).

A pulse may for example be described by a scaling factor. A pulse P itself may for example be described by a multiplication of a scaling factor F by the pulse profile S(t):P=F·S(t). The scaling factor F may be a scalar. The scalar may be a complex value, for example be dependent on a phase. The scaling factor F may for example function as a shim coefficient, for example for shimming a B1 field.

A pulse may for example be described by a length scaling factor. A pulse P itself may for example be described by a division of the time variable t by a length scaling factor T:P~S(t/T). The length scaling factor T may for example be a characteristic period of time for the pulse.

The pulse shape may for example be described with a time-bandwidth (TBW) product. Depending on the desired bandwidth, for example frequency bandwidth, for a fixed TBW product, this produces the temporal length T of the pulse. For example, with sinc pulses, for example those used for slice-selective excitation, the pulse shape may be described with a time-bandwidth product. For example, the temporal length T of the pulse corresponds to a number of zero crossings of a sinc pulse.

A pulse may for example be described by a scaling factor and a width scaling factor. A pulse P itself may for example be described by a multiplication of a scaling factor F by the pulse profile S(t) and by a division of the time variable t by a width scaling value T:P=F·S(t/T).

The pulse shape and/or amplitude and/or phase of an RF transmit pulse may for example correspond to a shape and/or amplitude and/or phase of a voltage pulse, that is applied to the respective transmit coil, and/or of a current pulse that is flowing through the transmit coil.

The excitation pulse may be a parallel excitation pulse and the at least one refocusing pulse is at least a parallel refocusing pulse. Parallel pulses are for example to be understood as pulses that are applied simultaneously over a number of transmit channels. For example, a parallel pulse may also be understood as a pTx pulse.

Advantageously the excitation pulse, for example the excitation pulse shape, is established as a function of the magnetic field distribution dataset, for example as a function of a B1 map, in such a way that a flip angle distribution generated by the excitation pulse (during a possible magnetic resonance measurement in accordance with the magnetic resonance sequence provided) is as homogeneous as possible. For each transmit channel, the excitation pulse shape, for example the excitation pulse profile, of the excitation pulse may be calculated for this on the basis of B1 maps and B0 maps. Gor each gradient coil of the gradient coil unit, a gradient pulse shape may be further calculated. Advantageously (during a magnetic resonance measurement) excitation pulses with the calculated excitation pulse shapes and gradient pulses with the calculated gradient pulse shapes are applied in parallel, for example in order to generate a flip angle distribution that is as homogeneous as possible.

The excitation angle for a pulse sequence may be referred to as the flip angle. It is usually the angle, related to the direction of the main magnetic field, in which a net magnetization is turned by application of an RF pulse at the Larmor frequency. In spin echo sequences for example a 90° pulse and a series of 180° pulses are used.

Such a technique is also known as B1 shimmung or RF shimming. Advantageously high-quality magnetic resonance images may be reconstructed from magnetic resonance signals that have been acquired during the presence of a homogeneous B1 field distribution.

Advantageously the excitation pulse, for example the excitation pulse shape, is established as a function of the magnetic field distribution dataset, for example as a function of a B1 map, in such a way that an SAR exposure brought about by the excitation pulse is as small as possible. Advantageously through this the load on a patient from the RF power absorbed during a magnetic resonance measurement is reduced.

Advantageously the excitation pulse may be more freely parameterized than the at least one refocusing pulse, since the pulse shape of the excitation pulse (by contrast with the pulse shape of the refocusing pulse) is not predetermined. The improved homogeneity of the complex flip angle distribution able to be achieved, that is generated at the beginning by the excitation pulse, then also acts positively on the spin echo following the at least one refocusing pulse (and possibly on stimulated echoes). For example, this improved spatial homogeneity of the magnetic resonance signal able to be achieved is retained over a number of spin echoes.

The predetermined refocusing pulse shape is for example independent of the magnetic field distribution dataset. The magnetic field distribution dataset provided thus has no influence on the refocusing pulse shape. The predetermined refocusing pulse shape is for example independent of an examination object to be examined by the magnetic resonance sequence provided. The refocusing pulse shape is defined for example in advance of a (diagnostic) magnetic resonance measurement, for example in a sequence development. Advantageously the refocusing pulse shape for the provision of the magnetic resonance sequence, for example for a specific magnetic resonance measurement in accordance with the magnetic resonance provided, does not need to be established again. This may bring about a significant saving in time, so that the provision of the magnetic resonance sequence may be carried out efficiently.

For example, a preliminary measurement for establishing the magnetic field distribution dataset may be carried out first of all with a magnetic resonance apparatus. For example, the data recorded in the preliminary measurement describes at least one part of the examination object and/or maps it. Then the magnetic resonance sequence may be provided, wherein the predetermination of the refocusing pulse shape, the establishment of the excitation pulse, the establishment of the at least one refocusing pulse and the provision of the magnetic resonance sequence may be carried out for example by a system control unit of the magnetic resonance apparatus. The system control unit may for example have a processor unit and/or a memory unit for this. In accordance with the magnetic resonance sequence provided, a main measurement may be carried out. The main measurement may be suitable for acquiring diagnostically evaluable and/or imaging magnetic resonance signals. With the aid of the magnetic resonance signals acquired in the main measurement one or more magnetic resonance images for example may be generated.

One embodiment of the method provides for the excitation pulse to be a dynamic pulse. An RF transmit pulse for example may be seen as a dynamic pulse, of which the phase and/or amplitude changes in the temporal course of the pulse, while an, for example prespecified, gradient trajectory is sampled by a gradient coil unit of the magnetic resonance apparatus. For example, the sampling of the gradient trajectory is carried out over time with the variation of phase and/or amplitude of the RF transmit pulse.

The totality consisting of RF transmit pulse and gradient trajectory may also be regarded as a dynamic pulse. The RF transmit pulse would then be a part of the dynamic pulse.

The at least one pulse shape and/or amplitude and/or phase of the RF transmit pulse or of a sub pulse may for example correspond to a shape and/or amplitude and/or phase of a voltage pulse that is applied to the respective transmit coil, and/or of a current pulse that is flowing through the transmit coil.

The at least one pulse shape and/or amplitude and/or phase of the gradient pulse may for example correspond to a shape and/or amplitude and/or phase of a voltage pulse that is applied to the gradient coil unit, and/or of a current pulse that is flowing through the gradient coil.

A dynamic pulse advantageously provides the B1 field generated by it to be controlled more precisely. For example, magnetic field inhomogeneities may be compensated for with a pTx pulse that, above all with higher field strengths of the main magnetic field as from 5, for example 7, tesla, may be especially advantageous. It is precisely at high field strengths that a dynamic parallel transmission may be of advantage, because the B1 effects occurring there are often so short-wave that they would be visible within a magnetic resonance image.

One embodiment of the method provides for the excitation pulse to be a pTx pulse. “pTx” stands for parallel transmission here. A pTx pulse may include a number of sub pulses, that are sent in parallel, for example at the same time, by one transmit coil of a radio-frequency antenna unit of the magnetic resonance apparatus in each case. Each transmit coil may in its turn be assigned to a transmit channel. In this case the sub pulses may for example differ in their pulse shape and/or amplitude and/or phase. The sub pulses may further have a time delay in relation to one another. For example, an RF transmit pulse able to be emitted consists of a number of sub pulses, that differ from one another and may each be sent by a transmit coil of a multi-channel transmit coil arrangement of the radio-frequency antenna unit. At least a part of the number of sub pulses, for example all sub pulses, may be dynamic pulses.

During sending of a pTx pulse a prespecified spatial distribution of the excitation is advantageously able to be achieved as an additional degree of freedom by interference of the signals of the number of transmit channels via a plurality of transmit coils of the radio-frequency antenna unit, that is set during establishment of the pTx pulse, by variation of phase and amplitude for example.

One embodiment of the method provides for the magnetic resonance sequence for a number of transmit channels to include one excitation pulse and at least one refocusing pulse in each case. There may be provision for the respective excitation pulse or refocusing pulse to be applied in parallel, for example at the same time.

The magnetic resonance sequence may be intended for example to be applied with a magnetic resonance apparatus, that includes N transmit channels. Preferably, in accordance with the magnetic resonance sequence, N excitation pulses (i.e. one excitation pulse per transmit channel), are applied in parallel, then in parallel N first refocusing pulses (i.e. one refocusing pulse per transmit channel) and possibly yet a further N refocusing pulses. This application of the N excitation pulses, N first (and if necessary further) refocusing pulses, is undertaken for example for each slice to be recorded.

The at least one refocusing pulse may be a static pTx pulse (“static” because it has a fixed pulse shape, so that only one B1 field is generated with it) and the excitation pulse may be a dynamic pTx pulse (“dynamic” because it has channel-specific pulse shapes, so that different B1 fields may constantly be generated during a pulse).

Static pTx may include all transmit coils being activated with the same RF pulse shape (for example rectangular or sinc shape), but scaling these with coil-specific magnitudes and phases. This enables a (static) excitation field to be generated, that may be spatially more homogeneous than an uncalibrated excitation field. Dynamic pTx preferably generates temporally varying B1 fields, each inhomogeneous per se, that are applied at the same time with likewise temporally varying B0 gradient fields, often described as “send k-space trajectory”. In this way very homogeneous flip angle distributions at the end of the dynamic pTx pulse may be achieved.

One embodiment of the method provides for the establishment of the excitation pulse shape to include an optimization of a homogeneity of a flip angle distribution able to be generated, for example to be generated, by the excitation pulse. The optimization may be undertaken with the aid of the magnetic field distribution dataset. The excitation pulse, for example its pulse shape, may be optimized with the aid of the at least one magnetic field distribution dataset in respect of a maximum homogeneity of the flip angle distribution. Advantageously any given pulse shape is available for optimization of the flip angle distribution. (By contrast, the at least one refocusing pulse has a predetermined pulse shape.)

Establishment of the excitation pulse shape may include an optimization of a homogeneity of the flip angle distribution in a slice (to be recorded). One embodiment provides for the establishment of the excitation pulse shape to include an optimization of a homogeneity of the flip angle distribution in just one, for example central, part region of a slice. Advantageously a part of the examination object's body to be recorded lies in this part region.

For example, the, for example dynamic, excitation pulse may be optimized so that it does not excite the whole slice, for example in the xy direction, but for example just a smaller rectangular section in the middle, in which for example a prostate of the patient lies. Then ideally nothing or just a little outside this is also excited by the refocusing pulses and does not then have to be spatially encoded. Advantageously in this way the part of the body to be recorded (without all that surrounds it) is recorded more quickly.

Advantageously during the excitation pulse, along with any further parameters, the pulse shape of the excitation pulse is also available as a parameter for the optimization of the homogeneity. (By contrast with this, the pulse shape of the at least one refocusing pulse is predetermined as fixed, i.e. would not be available for any optimization purposes.)

One embodiment of the method provides for excitation pulse and/or the at least one refocusing pulse to be spatially selective, for example slice-selective. The spatial selection may for example relate to a selection of a slice, but other spatial regions and/or geometries are also conceivable, for example a sphere. The excitation pulse shape and/or the refocusing pulse shape may include a Fourier transform, that corresponds to the desired spatial selection, for example to a desired local excitation profile.

The magnetic resonance sequence to be provided may provide for a selection gradient, for example slice selection gradients, to be applied at the same time as the excitation pulse and/or the at least one refocusing pulse. Advantageously a magnetic field gradient is generated by this in the examination area, so that in accordance with the frequency content of the excitation pulse and/or of the at least one refocusing pulse, a spatial region corresponding to this is excited.

Advantageously a pulse shape is predetermined as the refocusing pulse shape, that brings about a desired spatial selection, for example a slice selection. Advantageously the refocusing pulse shape has a spatially especially clearly defined effect. For example, a spatial region is predetermined, for example a slice or another volume, for which the refocusing pulse is to be selective.

If a magnetic field gradient is present, a specific spatial region, for example a specific slice, has a magnetic field strength B, that lies in a specific region of the magnetic field B, for example between Bₐ and B_{b}. Using the physical relationship f=γ·B, wherein γ is a core-specific gyromagnetic constant, the frequency f is linked to the magnetic field. Thus it is known which frequency content, for example frequency bandwidth, a corresponding RF pulse, for example the refocusing pulse, should have. Through a Fourier transform of frequency space or location space into the time space a pulse shape may be specified that corresponds as exactly as possible to a specified spatial region.

Advantageously a pulse shape, that corresponds to a specific excitation profile, for example a slice excitation, may be predetermined in advance as the refocusing pulse shape without a time-consuming recalculation for a magnetic resonance measurement already to be carried out being necessary.

When the excitation pulse is above all optimized to the extent of bringing about as high a homogeneity of the B1 field as possible, this ancillary condition may result in the spatial selection, for example slice selection, being deficient. Moreover, inaccuracies in the calibration (for example by imprecise B1 maps and/or B0 maps and/or through an imprecise calibration of the amplification of the transmit channels) cause such pulses to become prone to a poorer spatial selection.

Advantageously refocusing pulses with selection-optimized refocusing pulse shape “cut” the desired spatial profile, for example slice profile, out of a possibly deficient spatial profile, for example slice profile, of the excitation pulse and in this way ensure the desired selection, for example slice selection. Through the combination of an excitation pulse with free pulse shape and one or more refocusing pulses with predetermined pulse shape, that is optimized in respect of the excitation profile, for example a high B1 homogeneity with simultaneously precise spatial excitation may be achieved.

One embodiment of the method provides for the refocusing pulse shape to have a shape of a sinc pulse and/or of an SLR pulse and/or of a rectangular pulse.

The shape of a sinc pulse may for example be described mathematically by a sinc function (sinc(x)=sin (x)/x). Theoretically such a function would make a perfect slice selection possible. However an (unadapted) sinc pulse would require an infinite number of side lobes (and thus an infinite transmission time). Advantageously the sinc pulse is therefore modified, for example by a filter. For example the sinc pulses may be adapted by a window function, in order to reduce the pulse length to a desired extent. One possible window function is for example the Hamming window function.

SLR stands for “Shinnar-Le Roux”; SLR pulses may be established by an SLR algorithm. Advantageously SLR pulses have very good slice profiles with no side lobes and very small ripples outside the slice.

One embodiment of the method provides for the establishment of the at least one refocusing pulse to be carried out as a function of the magnetic field distribution dataset. The establishment advantageously includes an optimization of a homogeneity of a magnetic field distribution able to be generated, for example to be generated, by a refocusing pulse (for example B1 field distribution).

Even if the pulse shape of the at least one refocusing pulse is predetermined, other parameters of the refocusing pulse may advantageously be adapted to the magnetic field distribution dataset. Advantageously a better homogeneity of the magnetic field distribution able to be generated, for example to be generated, by the at least one refocusing pulse (for example B1 field distribution) may be achieved with this than without adaptation.

One embodiment of the method provides for the establishment of the at least one refocusing pulse to include an establishment of a scaling factor as a function of the magnetic field distribution dataset, wherein the refocusing pulse is established with the aid of the (predetermined) refocusing pulse shape and the scaling factor. For each refocusing pulse a separate vector with corresponding coefficients, for example one per transmit channel, may be calculated. Such a calculation may for example be undertaken with the DSC approach (“DSC” for direct signal control) described in Malik SJ, Beqiri A, Padormo F, Hajnal JV. Direct signal control of the steady-state response of 3D-FSE sequences. Magn Reson Med. 2015 March; 73 (3): 951-63. doi: 10.1002/mrm.25192. Epub 2014 Mar. 17. PMID: 24639096; PMCID: PMC7614097.

The scaling factor is preferably an, for example complex-value, scalar. For example, the scaling factor may be a coefficient of a mathematical representation of the refocusing pulse shape. For example, the scaling factor may represent a shim coefficient. The establishment of the refocusing pulse with the aid of the refocusing pulse shape and the scaling factor may for example include a multiplication of the scaling factor F_{RP} by a mathematical representation of the refocusing pulse shape, for example S_{RP}(t), for example P_{RP}=F_{RP}·S_{RP}(t).

One embodiment of the method provides for the magnetic resonance sequence to provide a sending of the excitation pulse and the at least one refocusing pulse by a number of transmit channels, wherein a channel-specific scaling factor is established for each transmit channel.

When the magnetic resonance sequence for example provides for a sending of the excitation pulse and of the at least one refocusing pulse by N transmit channels, N scaling factors are established, i.e. one scaling factor for each of the N transmit channels.

The channel-specific scaling factors may be represented for example in the form of a vector, for example of a column vector. For example, such a vector may include N coordinates, wherein each of the N coordinates includes a channel-specific scaling factor, for example consists of a channel-specific scaling factor.

One embodiment of the method provides for the at least one refocusing pulse to include a number of refocusing pulses. Advantageously a number of spin echoes are also generated by the number of refocusing pulses. Advantageously the better homogeneity able to be achieved by the excitation pulse may be retained across the number of spin echoes.

Advantageously a homogeneous flip angle and a corresponding phase of the nuclear spins is generated within a slice to be excited so that the following refocusing pulses, for example sinc-shaped, for example scaled with a suitable scaling factor, essentially generate the same phase profile.

Advantageously the scaling factors bring about a B1 shim or a shimmed B1 field. When for example all channel-specific scaling factors or shim coefficients are added to a 90° phase, the resulting B1 field also has a change by 90° in its spatial phase distribution. Advantageously the phase distribution brought about by the at least one refocusing pulse then differs from the phase distribution brought about by the excitation pulse by 90°.

For example, the magnetic resonance sequence may include a number of echo trains, wherein for each echo train an excitation pulse and at least one refocusing pulse is established in accordance with a method previously described.

The magnetic resonance sequence may be an RARE (rapid acquisition with relaxation enhancement) sequence, for example a TSE (turbo spin echo) sequence and/or an FSE (fast spin echo) sequence. Such sequences usually include one or more echo trains, each with one excitation pulse and a number of refocusing pulses.

A computer-implemented method for provision of an entire magnetic resonance sequence is further provided, that includes a number of magnetic resonance sequences provided in accordance with a previously described method for provision of a magnetic resonance sequence.

A magnetic resonance apparatus is further provided, that is configured to carry out a previously described computer-implemented method for provision of a magnetic resonance sequence or overall magnetic resonance sequence. The magnetic resonance apparatus includes a processing unit, for example including one or more processors, and/or a memory unit, for example including one or more memory modules, for carrying out the method.

A computer program product is further provided, that includes a program and is able to be loaded directly into a memory of a programmable system control unit of a magnetic resonance apparatus and has program means, for example libraries and auxiliary functions, for carrying out a provided method for providing a magnetic resonance sequence or overall magnetic resonance sequence when the computer program product is executed in the system control unit of the magnetic resonance apparatus. The computer program product may in this case include software with a source code, that is still to be compiled and linked or which only has to be interpreted, or an executable software code, that has only still to be loaded into the system control unit to be executed.

The computer program product provides for the method to be executed quickly, identically repeatedly and robustly. The computer program product may be configured so that it may execute the method steps by the system control unit. The system control unit in this case has the requirements, such as for example a corresponding main memory, a corresponding graphics card or a corresponding logic unit, so that the respective method steps may be carried out efficiently.

The computer program product is stored for example on a computer-readable medium or is held on a network or server, from where it may be loaded into the processor or a local system control unit, that may be directly connected to the magnetic resonance apparatus or be configured as part of the magnetic resonance apparatus. Furthermore, control information of the computer program product may be stored on an electronically readable data medium. The control information or the electronically readable data medium may be configured in such a way that, when the data medium is used in a system control unit of a magnetic resonance apparatus, it carries out the method.

Examples of electronically readable data media are a DVD, a magnetic tape or a USB stick, on which electronically readable control information, for example software, is stored. When this control information is read from the data medium and stored in a system control unit of the magnetic resonance apparatus, the methods previously described may be carried out.

Further advantages, features and details emerge from the embodiments described below and also with the aid of the drawings. Parts that correspond to one another are labeled with the same reference numbers in all figures.

### BRIEF DESCRIPTION OF THE FIGURES

FIG. 1 depicts a magnetic resonance apparatus.

FIG. 2 depicts an execution sequence of a method for provision of a magnetic resonance sequence according to an embodiment.

FIG. 3 depicts an echo train of a magnetic resonance sequence with an excitation pulse with free pulse shape and a number of refocusing pulses with predetermined pulse shape according to an embodiment.

FIG. 4 depicts a number of echo trains of an overall magnetic resonance sequence according to an embodiment.

FIG. 5 depicts a number of echo trains of a number of transmit channels according to an embodiment.

### DETAILED DESCRIPTION

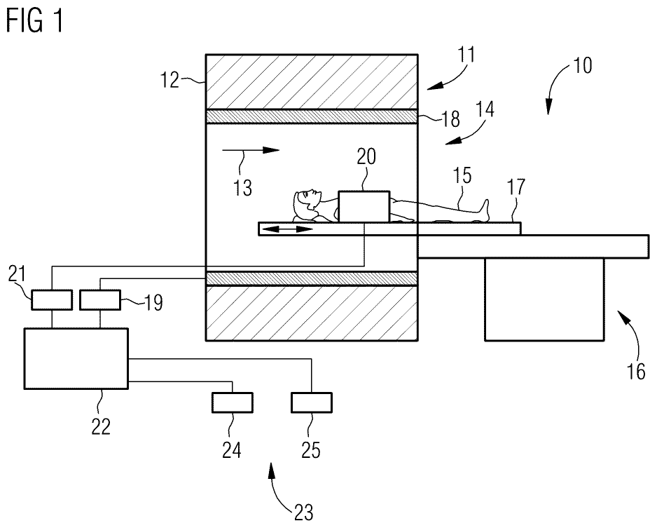

Shown schematically in FIG. 1 is a magnetic resonance apparatus 10. The magnetic resonance apparatus 10 includes a magnet unit 11, that includes a main magnet 12 for generating a strong and for example temporally constant main magnetic field 13, for example a B0 field. Moreover, the magnetic resonance apparatus 10 includes a patient receiving area 14 for receiving an examination object such as a patient 15. The patient receiving area 14 in the present embodiment is configured in a cylindrical shape and surrounded in a circumferential direction by the magnet unit 11. However an embodiment of the patient receiving area 14 that differs from this is conceivable. The patient 15 may be pushed by a patient support apparatus 16 of the magnetic resonance apparatus 10 into the patient receiving area 14. For this, the patient support apparatus 16 has a patient table 17 configured to be able to be moved within the patient receiving area 14.

The magnet unit 11 furthermore includes a gradient coil unit 18 for generating magnetic field gradients, that are used for spatial encoding during an imaging process. The gradient coil unit 18 is controlled by a gradient control unit 19 of the magnetic resonance apparatus 10. The magnet unit 11 furthermore includes a radio-frequency antenna unit 20, that in the present embodiment is configured as a component of a local coil 20. Local coils are usually arranged directly on the patient 15. The radio-frequency antenna unit 20 may however also be configured for example as a body coil permanently integrated into the magnetic resonance apparatus 10. It is precisely for magnetic resonance apparatuses that have a high main magnetic field strength, for example 7 tesla, that local transmit arrangements are often better suited.

The radio-frequency antenna unit 20 is controlled by a radio-frequency antenna control unit 21 of the magnetic resonance apparatus 10 and radiates RF pulses into an examination space, that is essentially formed by a patient receiving area 14 of the magnetic resonance apparatus 10. Through this an excitation of atomic nuclei takes place in the main magnetic field 13 generated by the main magnet 12. By relaxation of the excited atomic nuclei magnetic resonance signals are generated. The radio-frequency antenna unit 20 is configured for receiving the magnetic resonance signals.

The radio-frequency antenna unit 20 may include a number of transmit coils, for example coil elements, that are each configured to generate an RF pulse; such an RF pulse may for example be a sub pulse of a dynamic pulse and/or pTx pulse. Each of these number of transmit coils may be assigned to a transmit channel. Such a transmit channel may for example further include transmit channel-specific components such as for example a signal amplifier. Such components may for example be an element of the radio-frequency antenna control unit 21.

For control of the main magnet 12, of the gradient control unit 19 and for control of the radio-frequency antenna control unit 21 the magnetic resonance apparatus 10 has a system control unit 22. The system control unit 22 centrally controls the magnetic resonance apparatus 10, such as for example the carrying out of an imaging magnetic resonance sequence, for example of a spin echo sequence. Moreover, the system control unit 22 includes an evaluation unit, not shown in any greater detail, for evaluation of the magnetic resonance signals that are acquired during the magnetic resonance examination. Furthermore, the magnetic resonance apparatus 10 includes a user interface 23, that is connected to the system control unit 22. Control information, such as for example imaging parameters, as well as reconstructed magnetic resonance images, may be shown on a display unit 24, for example on at least one monitor, of the user interface 23 for a medical operator. Furthermore, the user interface 23 includes an input unit 25, by which information and/or parameters may be entered by the medical operator during a measuring process.

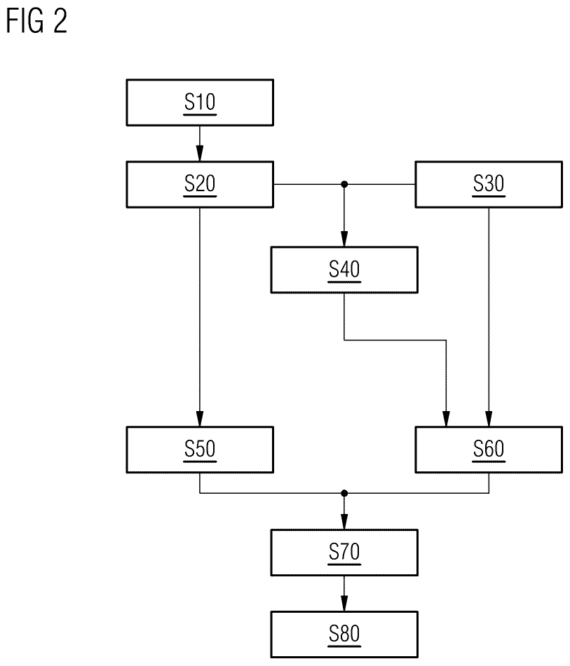

Shown in FIG. 2 is a possible execution sequence of a computer-implemented method for provision of a magnetic resonance sequence, for example of a spin-echo sequence. The magnetic resonance sequence provided by this may be carried out for example with a magnetic resonance apparatus 10 shown in FIG. 1.

In accordance with the form of embodiment shown, in S10 a magnetic field distribution dataset is recorded with the magnetic resonance apparatus 10 and is provided in S20. The magnetic field distribution dataset may for example include a B1 field map and/or a B0 field map.

In S30 a refocusing pulse shape is predetermined. Preferably in S40, for at least one of the refocusing pulses, for example for each refocusing pulse, a scaling factor is established as a function of the magnetic field distribution dataset provided in S20. The refocusing pulse is then preferably established in S50 with the aid of the refocusing pulse shape predetermined in S30 and the scaling factor established in S40.

If the magnetic resonance sequence provides for sending of an excitation pulse and of the at least one refocusing pulse by the number of transmit channels, for example if the radio-frequency antenna unit 20 includes a number of transmit coils, a channel-specific scaling factor may be established for each transmit channel. A refocusing pulse includes a number of channel-specific sub pulses, that are intended to be sent in parallel, for example at the same time.

In S50 an excitation pulse is established, that has an excitation pulse shape, wherein the excitation pulse shape is established as a function of the magnetic field distribution dataset. The excitation pulse may be a dynamic pulse and/or pTx pulse. In S50, the excitation pulse may be optimized in respect of a homogeneity of a magnetic field distribution and/or an SAR exposure by the excitation pulse able to be generated by the excitation pulse. The excitation pulse may be spatially selective, for example slice-selective. For example a specific region, for example a specific slice, within the patient receiving area 14 or the patient 15 is excited by the excitation pulse.

In S60 at least one refocusing pulse is established, that has the refocusing pulse shape predetermined in S30. The at least one refocusing pulse may include a number of refocusing pulses, i.e. a number of refocusing pulses are generated in S60; the number of refocusing pulses may be applied one after the other in the magnetic resonance sequence; advantageously an echo train may be generated with this. The magnetic resonance sequence may be an RARE (rapid acquisition with relaxation enhancement) sequence, for example a TSE (turbo spin echo) sequence and/or an FSE (fast spin echo) sequence.

The at least one refocusing pulse may be spatially selective, for example slice-selective. For example a specific region, for example a specific slice, within the patient receiving area 14 or the patient 15 is excited by the refocusing pulse. The refocusing pulse shape predetermined in S30 may be optimized to the extent of bringing about the desired spatial selection. For example the refocusing pulse shape has the shape of an, for example adapted, sinc pulse and/or of an SLR pulse. These pulses are particularly well suited to exciting a spatially precisely defined slice profile.

In S70 the magnetic resonance sequence with the excitation pulse and the at least one refocusing pulse is provided. The magnetic resonance sequence may for example include one excitation pulse and at least one refocusing pulse for a number of transmit channels.

S20, S30, S40, S50, S60 and S70 may for example be carried out by the system control unit 22 of the magnetic resonance apparatus 10. In S80 the magnetic resonance apparatus 10 carries out a magnetic resonance measurement in accordance with the magnetic resonance sequence provided in S70.

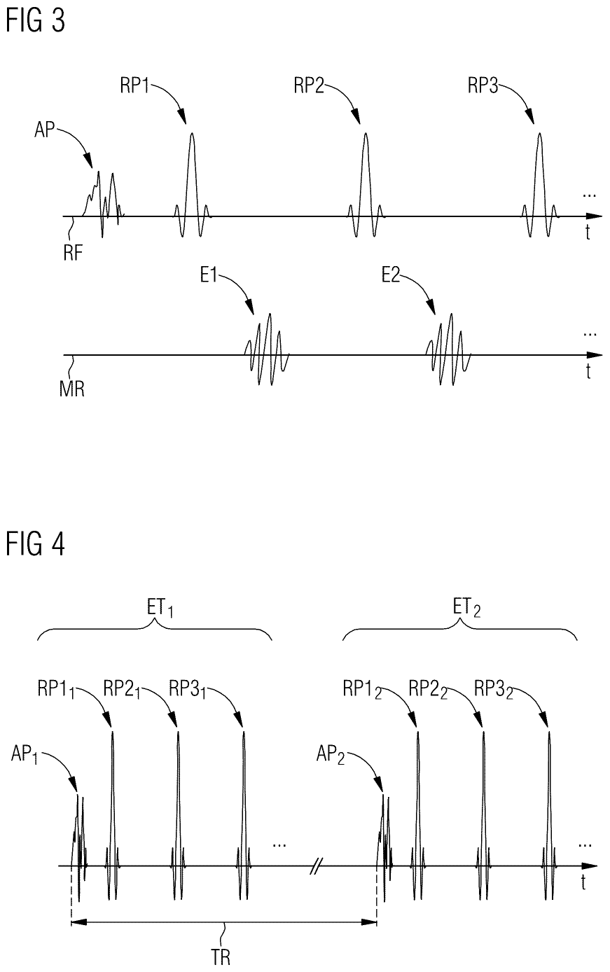

Various possible aspects of the method for provision of the magnetic resonance sequence are intended to be explained in greater detail with the aid of the sequence diagrams of FIGS. 3, 4 and 5. The elements of the magnetic resonance sequences are arranged here on a timeline t.

The excitation pulse established in S50 may be a slice-selective, dynamic pTx excitation pulse, that is parameterized even more freely than the conventional pulses. For example such an excitation pulse AP is shown in FIG. 3. It is followed in time by three refocusing pulses RP1, RP2 and RP3, that have a predetermined pulse shape, that in this example has the shape of a sinc function. A further pulse shape may for example be that of an SLT pulse. The refocusing pulses RP1, RP2, RP3 are combined with the excitation pulse AP to form a magnetic resonance sequence or pulse sequence. The magnetic resonance sequence may also have yet more refocusing pulses.

Shown in FIGS. 3, 4 and 5 are only the, for example real-value, momentary values of the pulses or pulse profiles. It is further conceivable for the pulses or pulse profiles to have a phase, for example a phase differing from zero. The pulses or pulse profiles may then be described for example by a complex-value function.

The refocusing pulses RP1, RP2 and RP3 bring about a reversal of the phase of the nuclear spins excited by the excitation pulse AP. Spin echoes E1, E2, . . . , that may be recorded as magnetic resonance signals by the radio-frequency antenna unit 20, are generated by this.

The refocusing pulses RP1, RP2 and RP3 may be “simply shimmed” in accordance with a “direct signal optimization”; in a “direct signal optimization” a B1 shim or a B1 field is calculated and applied for each pulse in the pulse sequence. In this way the homogeneity may be improved; however the possible improvement is limited by the fact that an individual pulse may also only be optimized in respect of a single B1 field. The overall homogeneity, for example of the generated flip angle, is thereby not usually at the high level of dynamic pTx pulses.

The excitation pulse AP may be a free, dynamic pTx pulse, that may consist of any given pulse shapes and also for example gradient pulse shapes. (On the other hand the refocusing pulses RP1, RP2 and RP3 are form-bound and consist for example of one or more sinc-formed RF sub pulses, that thus have a predetermined pulse shape, paired with slice-selection gradients.) A free, dynamic pTx pulse, that consists of any given RF pulse shapes, makes possible more different B1 and gradient fields (degrees of freedom for optimization) and is for example not bound to the sinc forms. Thus a homogeneous excitation with little SAR within the slice may advantageously be generated. Because of any imprecisions in the calibration (for example in respect of RF power amplifiers of the radio-frequency antenna unit 20, the B1 fields and/or the B0 fields), such pulses are however prone to worse slice profiles.

Advantageously, through the excitation pulse AP, a homogeneous flip angle and a corresponding phase are generated in any event within the slice to be excited in this case so that subsequent sinc-shaped refocusing pulses RP1, RP2 and RP3 with a specific B1 shim may generate the same phase profile (+90°=difference between the phase of the excitation pulse and the refocusing pulse). Thereby it may be sufficient to carry out a direct signal optimization only for the refocusing pulses. The better homogeneity, that is generated at the beginning by the excitation pulse AP, is retained over a number of echoes.

The refocusing pulses EP1, EP2, EP3 advantageously “cut” an exact slice profile out of the possibly inexact slice profile of the excitation pulse AP and in this way ensure the desired slice selection. By themselves being short and not dynamic pTx pulses, they advantageously make possible a robustness against B0 inhomogeneities, movement and reduce T2 blurring. A combination of the described refocusing pulses EP1, EP2, EP3 with the described excitation pulse AP advantageously leads to a marked improvement of the homogeneity and to less SAR exposure by comparison with pure direct signal optimization.

In FIG. 4 it is shown that a magnetic resonance sequence may also include a number of echo trains ET₁, ET₂, each of which includes an excitation pulse AP₁ or AP₂ and a number of refocusing pulses RP1₁, RP2₁, RP3₁ or EP1₂, EP2₂, EP3₂. The excitation pulses AP₁ and AP₂ have a repetition time (TR).

AP1, RP1₁, RP2₁, RP3₁ may be expressed as a first magnetic resonance sequence and AP₂, RP1₂, RP2₂, RP3₂ as a second magnetic resonance sequence, that are part of an overall magnetic resonance sequence. Thus the first magnetic resonance sequence and the second magnetic resonance sequence (and any further magnetic resonance sequences) may be provided in accordance with a method shown in FIG. 2.

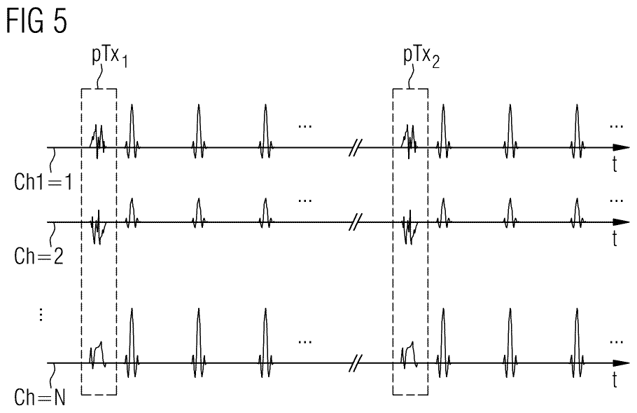

Shown in FIG. 5 are a number of echo trains of a number of transmit channels of the magnetic resonance apparatus 10. At the beginning of an echo train an excitation pulse is applied in each case in parallel on each of the transmit channels Ch=1, 2, . . . N, that together may be expressed as pTx pulses pTx₁, pTx₂. For example the excitation pulse of the channel Ch=1 may be expressed as first sub pulse, the excitation pulse of the channel Ch=2 as second sub pulse, . . . , and the excitation pulse of the channel Ch=N as Nth sub pulse of a pTx pulse pTx₁, pTx₂. In this case each of the N sub pulses may have another pulse shape. The pulse shape is for example dependent on the magnetic field distribution dataset provided in S20. The pulse shape of the refocusing pulses following on from the excitation pulses (scaled per transmit channel) is always the same on the other hand, because it is predetermined as fixed.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for providing a magnetic resonance sequence, the method comprising:
acquiring a magnetic field distribution dataset;
determining a refocusing pulse shape;
computing an excitation pulse that has an excitation pulse shape, wherein the excitation pulse shape is computed as a function of the magnetic field distribution dataset;
computing of at least one refocusing pulse that has the predetermined refocusing pulse shape; and
providing the magnetic resonance sequence with the excitation pulse and the at least one refocusing pulse.

2. The method of claim 1, wherein the magnetic resonance sequence is a spin echo sequence.

3. The method of claim 1, wherein the excitation pulse is a parallel excitation pulse and the at least one refocusing pulse is an at least one parallel refocusing pulse.

4. The method of claim 1, wherein the excitation pulse is a dynamic pulse.

5. The method of claim 1, wherein the magnetic resonance sequence for a number of transmit channels comprises the excitation pulse and the at least one refocusing pulse in each number of transmit channels.

6. The method of claim 1, wherein the magnetic field distribution dataset comprises a B1 field map.

7. The method of claim 1, wherein computing the excitation pulse shape comprises an optimization of a homogeneity of a flip angle distribution able to be generated by the excitation pulse and/or an optimization of an SAR exposure by the excitation pulse.

8. The method of claim 1, wherein computing the excitation pulse shape comprises an optimization of a homogeneity of a flip angle distribution in only one part region of a slice.

9. The method of claim 1, wherein the excitation pulse and/or the at least one refocusing pulse are spatially selective.

10. The method of claim 1, wherein the refocusing pulse shape has a shape of an adapted, sinc pulse and/or of an SLR pulse and/or of a rectangular pulse.

11. The method of claim 1, wherein the at least one refocusing pulse is computed as a function of the magnetic field distribution dataset.

12. The method of claim 1, wherein computing of the at least one refocusing pulse comprises computing a scaling factor as a function of the magnetic field distribution dataset and wherein the refocusing pulse is computed based at least in part on the refocusing pulse shape and the scaling factor.

13. The method of claim 12, wherein the magnetic resonance sequence provides for a sending of the excitation pulse and of the at least one refocusing pulse by a number of transmit channels and wherein a channel-specific scaling factor is computed for each transmit channel.

14. The method of claim 1, wherein the at least one refocusing pulse comprises a number of refocusing pulses.

15. The method of claim 1, wherein the magnetic resonance sequence is a rapid acquisition with relaxation enhancement (RARE) sequence.

16. The method of claim 15, wherein the RARE sequence comprises a turbo spin echo (TSE) sequence and/or an fast spin echo (FSE) sequence.

17. A system comprising:
a magnetic resonance apparatus configured to acquire a magnetic field distribution dataset; and
a control unit configured to determine a refocusing pulse shape, compute an excitation pulse that has an excitation pulse shape, wherein the excitation pulse shape is computed as a function of the magnetic field distribution dataset, and compute of at least one refocusing pulse that includes the predetermined refocusing pulse shape, and provide the magnetic resonance sequence with the excitation pulse and the at least one refocusing pulse to the magnetic resonance apparatus.

18. A non-transitory computer readable storage medium comprising a set of computer-readable instructions stored thereon, the computer-readable instructions that, when executed by a system control unit of the magnetic resonance apparatus cause the system control unit to:
acquire a magnetic field distribution dataset;
determine a refocusing pulse shape;
compute an excitation pulse that has an excitation pulse shape, wherein the excitation pulse shape is computed as a function of the magnetic field distribution dataset;
compute of at least one refocusing pulse that includes the predetermined refocusing pulse shape; and
provide the magnetic resonance sequence with the excitation pulse and the at least one refocusing pulse.
